(19) United States
(12) Patent Application Publication
(10) Pub. No.: US 2020/0005928 A1
(43) Pub. Date: Jan. 2, 2020

(54) SYSTEM AND METHOD FOR PERSONALIZED WELLNESS MANAGEMENT USING MACHINE LEARNING AND ARTIFICIAL INTELLIGENCE TECHNIQUES

(71) Applicant: GOMHEALTH LLC, Madison, NJ (US)

(72) Inventor: Sunil Daniel, Madion, NJ (US)

(21) Appl. No.: 16/452,735

(22) Filed: Jun. 26, 2019

Related U.S. Application Data
(60) Provisional application No. 62/690,585, filed on Jun. 27, 2018.

(51) Int. Cl. G16H 20/60; G06N 20/00

(52) U.S. Cl. CPC G16H 20/60; G06N 20/00

(57) ABSTRACT

The disclosure generally relates to a system and method for generating personalized wellness plans for users and monitoring adherence to such plans while providing real-time feedback to users. The personalized wellness plans are generated based on machine learning and/or artificial intelligence techniques and can provide guidance for weight loss, exercise, behavior and lifestyle modification, and integrative health and mindfulness.

### CROSS-REFERENCE TO RELATED APPLICATIONS

This application claims priority to and the benefit of U.S. provisional patent application with Ser. No. 62/690,585 filed on Jun. 27, 2018, entitled “Use of Machine Learning and AI Techniques to Provide Personalized Obesity Management and Feedback to Users”.

### BACKGROUND

### Field of the Invention

The present invention relates generally to the field of providing personalized health management to users, particularly for users suffering from obesity and obesity-related medical conditions, in order to enhance personal wellness through science and technology-based lifestyle, dietary, and activity planning and monitoring.

### Background Information

Americans often struggle to lose weight, and to maintain any weight loss over the long term. At any given time, more than 100 million Americans are trying to lose weight, and are spending $150 billion doing so. The unfortunate reality is that more than 80% of these people will gain back any weight that is lost in 3-12 months.

Although a number of weight-loss solutions are offered in the marketplace, none employ state of the art technology. Additionally, the time demands associated with many of the most popular programs available (e.g., making weekly appointments, traveling to counseling sessions, parking at the session location, etc.) are difficult to manage given the busy lives of many ordinary Americans.

Conventional systems do not provide the level of personalization that is required to provide optimal guidance for weight loss. In contrast, such conventional systems utilize generalized settings and suggestions that are not based on actual user habits, lifestyle choices, and preferences, nor which are based on an individual's specific wellness and weight loss requirements and goals.

Thus, there is a need for a system that provides real-time monitoring and feedback to provide lifestyle guidance in order to optimize weight loss, as well as obesity and diabetes management in an efficient and user-friendly manner.

### SUMMARY

Some embodiments of the invention are directed to employing state of the art technology for real-time tracking and feedback based on multiple variables (such as, but not limited to, environment, genetics, physiology, phenotype, behavior, and anthropometry). Some embodiments of the invention may measure changes in body weight in response to varying treatment substantially in real-time. For example, some embodiments of the invention provide an overall methodology for helping users (e.g., patients) manage obesity and weight loss. For example, some embodiments are directed to helping patients to create an overall health vision, and to clearly define personal values, barriers and challenges present in their lives, according to leading-edge techniques. This and other information may be used to create personalized nutrition, behavior and activity plans to help individual users achieve their goals. In some embodiments, users may be assigned a coach and/or other resources for assistance and support.

Some embodiments of the invention are directed to putting the methodology into practice via a technological platform. For example, some embodiments may include components for collecting any of various forms of data from users, healthcare providers, coaches and/or other data sources, and for applying any of various types of decision-making algorithms (e.g., predictive models, machine learning procedures, artificial intelligence, and/or any other suitable types of decision-making algorithms) to identify and generate meaningful feedback to individual users to keep them on track. In some embodiments, feedback may be delivered via wearable and/or other digital devices, in real time as events occur in users' lives. Data on any actions taken by the user in response to the feedback may be processed and used in modifying the user's tailored plans and in providing appropriate further feedback.

By putting the methodology into practice via the platform, some embodiments of the invention may provide an innovative, user-centric and evidence-based solution for obesity and weight management, which uses cutting-edge technology to deliver meaningful, real-time feedback to users, when and where it is needed.

### BRIEF DESCRIPTION OF THE DRAWINGS

These and other embodiments of the disclosure will be discussed with reference to the following exemplary and non-limiting illustrations, in which like elements are numbered similarly, and where:

FIG. 1 is a flowchart of a representative process whereby a user interacts with a platform for weight management, in accordance with some embodiments of the invention;

FIG. 2 is an architecture diagram of the platform, in accordance with some embodiments of the invention;

FIG. 3 is a flowchart of a representative process whereby a platform for managing weight loss provides nutrition-related feedback to a user using GPS location data;

FIG. 4 is a flowchart of a representative process whereby a platform for managing weight loss provides nutrition-related feedback to a user using GPS location data and the user's hunger hormone levels;

FIG. 5 is a block diagram depicting a representative computing system that may be used to implement aspects of some embodiments of the invention;

FIG. 6 is a flowchart for generated a personalized wellness plan and monitoring user adherence to the plan, in accordance with some embodiments of the invention;

FIG. 7 is a diagram depicting the various aspects of personalized wellness plan; and

FIG. 8 is a chart comparing an embodiment of the present invention with a traditional clinical approach and a traditional online approach.

### DETAILED DESCRIPTION

It should be understood that aspects of the invention are described herein with reference to the figures, which show illustrative embodiments. The illustrative embodiments herein are not necessarily intended to show all embodiments in accordance with the invention, but rather are used to describe a few illustrative embodiments. Thus, aspects of the invention are not intended to be construed narrowly in view of the illustrative embodiments.

Some embodiments of the invention are directed to a methodology for obesity and weight loss management, and to a technological platform for putting the methodology into practice. For example, some embodiments may include components for collecting any of various forms of data from users, healthcare providers, coaches and/or other data sources, and for applying any of various types of decision-making algorithms (e.g., predictive models, machine learning procedures, artificial intelligence, and/or any other suitable types of decision-making algorithms) to the data to identify and generate meaningful feedback to individual users to keep them on track to achieving their goals. In some embodiments, the platform may deliver feedback via devices which are worn or transported by users, in real time as events occur in the users' lives. Data on any actions taken by the user in response to the feedback may be processed by applying decision-making algorithms to identify changes to users' tailored plans to help them stay on track.

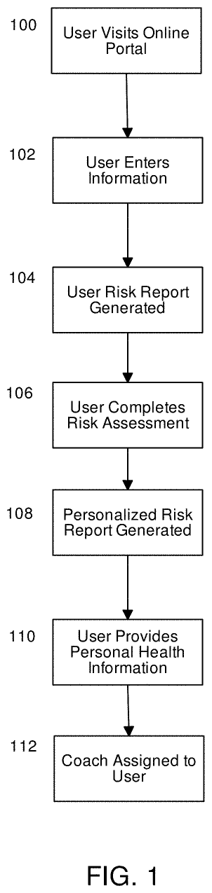

FIG. 1 depicts a high-level process by which a user is initially set up on an obesity and weight management platform, in accordance with some embodiments of the invention. Although the initial setup process is often performed by a user, it should be appreciated that other users of the platform may include healthcare providers, coaches, and/or any other suitable actors.

At step 101, the accesses a portal, such as a website or software application on their mobile device, and in step 102, the user provides certain specified information to the website. In the example shown, this information includes basic demographic information such as age, sex, ethnicity, etc. Next, the user accepts a disclaimer and terms and conditions specified by the website. As a result of the user's acceptance, in step 104 free “risk report” relating to weight loss management is generated, based upon the demographic information which the user supplied in 102. The “risk report” can be based on, for example, aggregated data, statistics, outcomes, and weight loss and management results from users having similar demographic profiles.

Next, the user clicks on a link providing access to a more detailed risk report. The user can set up an account if one has not already been established, such as by specifying a user name and password. The user then provides further information, such as their contact and billing information. The account registration process is now completed.

In step 106, the user completes a risk assessment questionnaire, which can be partially based upon the user's previous input in step 102. For example, if data supplied by the user satisfies certain criteria (which may be determined in any suitable fashion), then the user may be asked more detailed questions so as to assess obesity- and weight-related risks. Such questions can relate to, for example, the user's lifestyle choices, which may include fitness and exercise activity, dietary choices, sleep activity, water consummation activity, and the like. Using this information, a personalized disease risk report is generated for the user in step 108. The personalized disease risk report can be generated, for example, using aggregate data matching the user demographic information as well as lifestyle choices. The user is then prompted to approve paid access to the platform going forward. For example, the user may be asked to pay for access to the platform for a six-month period during which personalized treatment is to be provided (and again for a maintenance plan (e.g., in six month increments)). To continue with the offered services, the user pays for the program via a payment portal.

In step 110, the user completes a proprietary questionnaire to provide information which may be used during treatment, such as personal health information, medications, medical records, medical histories, hospitalization records, etc. This information is used to completes an integrative health, mindfulness-based, 4-step interactive process with the user to form an overall health vision and a health value statement. In step 112, a coach is assigned to the user, and an initial meeting is scheduled to initiate a weight loss management program. The coach can be selected based on a matching algorithm that accounts for a coach's experience and history with users having similar demographics as the user, as well as the user's preference for a particular type of coach based on gender, age, and/or location.

In some embodiments, the platform on which a user may be initially set up using the process of FIG. 1 may enables a personalized plan to be generated, based upon each individual's user's personalized obesity and weight management needs. In some embodiments, input supplied by a user is used to establish goals, such as by comparing the user's input to predetermined criteria (e.g., reflected in data stored in a database). One or more tailoring algorithms may use phenotypic input to establish a program plan for the user with respect to nutrition, behavior and activity. In some embodiments, genetic and/or microbiome input may also, or alternatively, be used to establish nutrition, behavior and/or activity plans. An integrative health methodology may be used to assess the user's state of readiness to change.

In addition, data from a fitness tracking device or software application can be input into the system, for example, from a FitBit™ or an Apple Watch™. A user's sleep data, such as sleep times, sleep patterns, REM and non-REM sleep data can also be input using a sleep tracking device or software application.

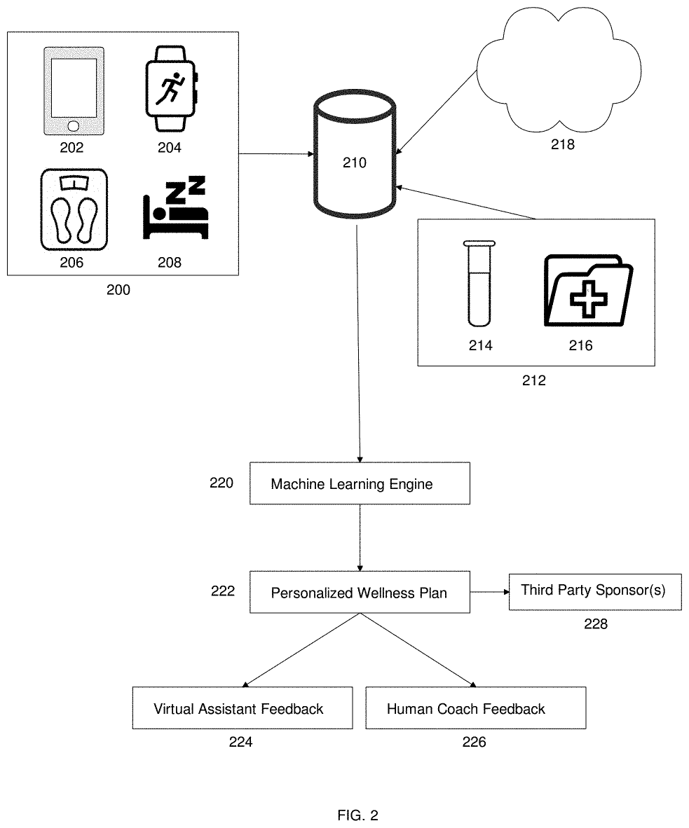

FIG. 2 is an architecture diagram of the platform, in accordance with some embodiments of the invention. Data sources 200 can includes, for example, data from a user's mobile computing device 202, smart watch 204, smart scale 206, and sleep tracking devices or systems 208. The data sources 200 can also include manual data input by the user, such as via meal and calorie tracking applications, and activity and workout tracking applications. A database 210, such as a cloud-based database, virtual database, or a physical database, receives information and records in real-time, or in pre-determined intervals, from the data sources 200.

In addition, the user's medical data 212, such as lab results 214 and electronic health records 216, can be transmitted to the database 210. The medical data 212 can be accessed via, for example, an application programming interface (API) with a medical facility, electronic health record providers, or clinical laboratory.

The database 210 can also receive aggregated data 218 from third-party users who may or may not have similar physiological or demographic traits and characteristics as the user. For example, if the user is a 50-year old male with diabetes, the system can aggregate anonymous physiological, medical, dietary, sleep, activity, and weight data from other users which are within a threshold of the user's age, and who also have been diagnosed with diabetes.

The data stored in the database 210 can be accessed by a machine learning engine 220 that processes the data. The machine learning engine 220 can utilize a variety of techniques, such as supervised learning, unsupervised learning, semi-supervised learning, and reinforcement learning to generate a personalized user wellness plan, as well as to track user developments and provide personalized user feedback.

The personalized user wellness plan 222 can be shared with various third-party sponsors 228, such as the user's friends and family who can provide additional support and encouragement to the user, insurance providers, medical providers, employers, and other entities and individuals who may be involved in the user's care and treatment, such as dieticians, nutritionists, and personal trainers.

In an embodiment, the system can provide anonymous, non-personal data to third-party marketers, advertisers, pharmaceutical companies, research institutions, and government agencies.

The personalized user wellness plan is then communicated as discussed in more detail below with the user, via a virtual assistant feedback 224 that utilizes the user's wearable or mobile computing device, and/or via a human coach feedback 226 that provides in-person, video, or telephone support and encouragement.

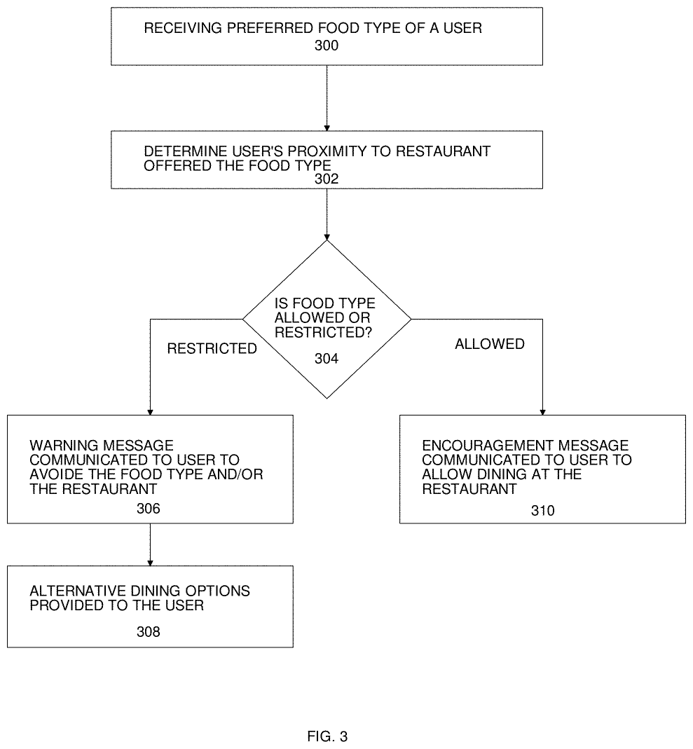

FIG. 3 is a flowchart of a representative process whereby a platform for managing weight loss provides nutrition-related feedback to a user using GPS location data. In the example illustrated, the user is, for example, a 55 year old professional male who takes the train to work in New York City daily, who is exposed daily to his favorite bagel shop on the way to work, and often complains of hunger. The user's lab results show an increase in hunger hormone recently, and despite exercising regularly, his metabolism has dropped. He slowly starts craving a bagel and cream cheese for breakfast instead of the two boiled eggs, cheese and coffee prescribed for him.

In step 300, a preferred food type of the user is determined, as described in more detail above. Next, in step 302, the user's movements, commute, and activity can be monitored using GPS-enabled technology within a wearable device, such as a fitness tracker, smart watch or smart glasses, or a mobile computing device, such as the user's smartphone, PDA, or tablet. The GPS location data can be collected over time and analyzed by the system using machine learning to identify travel patterns, timings, and visits to, for example, particular restaurants or dining establishments.

In the scenario described above, the system can determine, based on the GPS location data if a user is predisposed to eating at a restaurant that offers a food that the user prefers, but which has been deemed as a restricted food. In step 304, the system determines if the food is an allowed food or a restricted food. If the food is a restricted food, then in step 306, the system can transmit a warning message to the user, instructing or encouraging the user to dine at another restaurant, or to avoid eating the restricted food.

In addition, in step 308, alternative dining options can be presented to the user, such as nearby restaurants, or restaurants along the user's route or commute, which do not include the restricted food or food type.

In an embodiment, the warning message can also include a reminder or reinforcement of the user's weight loss and/or health goals, and can provide a status update or show the progress made towards reaching the goals.

Alternatively, if the system determines that the preferred food type is allowed, and is available at the restaurant, then, in step 310, the system can provide an encouragement message informing that the user that it is “okay” to eat the preferred food.

The warning and encouragement messages can be delivered to the user's device via a voice call, video call, text message, MMS message, or via haptic feedback. In an example, a human coach makes an audio or video call to the user to provide the message.

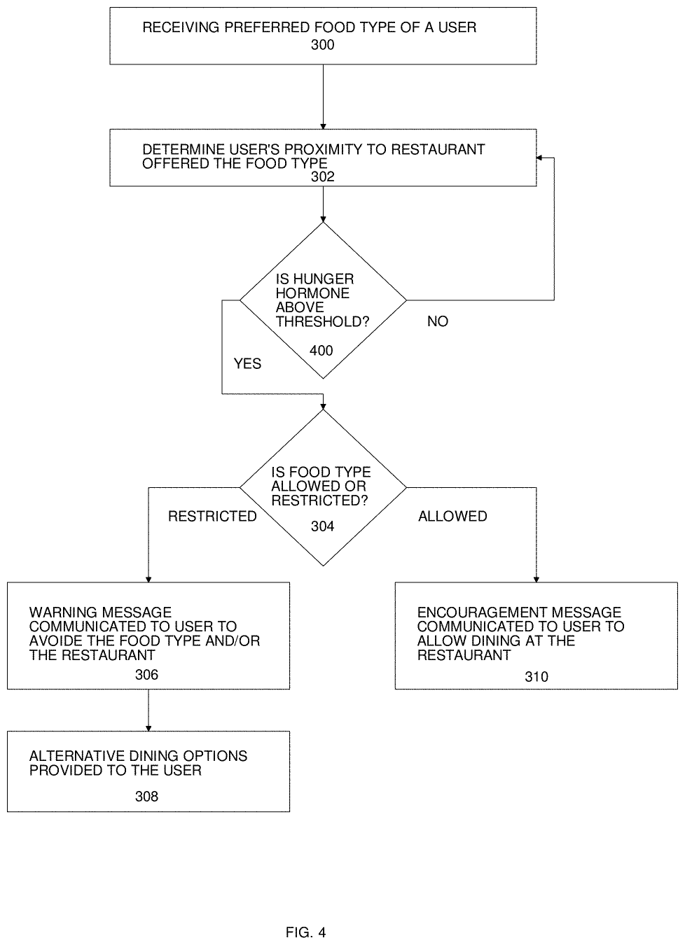

FIG. 4 is a flowchart of a representative process whereby a platform for managing weight loss provides nutrition-related feedback to a user using GPS location data and the user's hunger hormone levels. In step 400, the system receives data related to the hunger hormone level of the user, and utilizes the hunger hormone level data in conjunction with a user's proximity to a restaurant that offers a restricted food or food type. If the hunger hormone is above a certain threshold level, which may indicate a user's increased appetite or propensity to deviate from their personalized nutritional plan, the process continues to step 304 as described above.

The hunger hormone levels can be based on user's laboratory tests, or alternatively, can be based on manually entered (i.e., self-reported) hunger and appetite levels by the user. For example, the user can enter their hunger level on a scale from “1” to “10”, with “1” being not hungry to “10” being extremely hungry. In addition, the hunger level can be entered as a phrase, such as “slightly hunger”, “starving”, “normal hunger”, etc.

If the hunger hormone is not above a certain threshold level, then the process reverts back to step 302, where the system continually monitors the user's proximity to a restaurant that offers a preferred food type of the user.

For example, over a six-month timeline, data can indicate that, through week 24, the user was on track to achieve his goals, was motivated and complied with the personalized nutrition, behavioral and activity plan developed for him. Since then, however, he has begun to struggle, experienced increases in stress, and decreases in free time. Perhaps unsurprisingly, he has started to regain weight.

In accordance with the techniques described herein, one or more decision-making algorithms may be applied to data gathered on the user to discern his struggles maintaining weight loss, identify his trends and patterns, and compare those trends and patterns to those of other users in his specific cohort. Various types of information that may be collected on the user over time, such as data on the user's weight (e.g., provided by a wireless scale, indicating acute weight loss from the start of the period until week 24), data indicating the user's hunger hormone level (indicating that the hormone was at or near a baseline level until week 24) and or satiety level, and data on the user's activity level, compliance, goal achievement, coaching intensity, and metabolic rate may be processed. The hunger hormone can include, for example, ghrelin, leptin, cortisol, glucose, insulin, neuropeptide Y (NPY), agouti-related protein (AgRP), proopiomelanocortin, alpha-melanocyte stimulating hormone (α-MSH), cocaine- and amphetamine-regulated transcript (CART), cholecystokinin, peptide tyrosine tyrosine (PYY), pancreatic polypeptide (PP), oxyntomodulin, glucagon-like peptide 1 (GLP-1), gastric inhibitory polypeptide (GIP), and adiponectin. By applying one or more decision-making algorithms to this information, it may be determined whether the user has achieved his/her goal and is adhering to previous recommendations. If the user has achieved his/her goal, the system may congratulate the user and provide guidance on how to stay the course going forward. If the user has not achieved his/her goal but is adhering to previous recommendations, then the system may provide positive feedback and highlight a predicted timeline to achieve the goal, and revise an existing personalized plan as appropriate. If the user is not adhering to previous recommendations, then the system may alter the user's personalized plan and predict a new timeline to achieve the user's goal. Any of various modifications to the user's nutritional, activity and/or behavioral plans may be developed, and the user and/or his coach may be informed of a new or updated plan so that it may be implemented. Further monitoring of the user's progress may enable him to achieve his end goals at the end of the program (e.g., continued weight loss, maintenance of current weight, prevent regain, etc.).

It should be appreciated from the foregoing that the methodology and platform disclosed herein offers a number of advantages over prior approaches to obesity and weight loss management. For example, some embodiments of the invention are directed to the creation of tailored nutritional, activity and behavioral plans based upon information which includes user phenotype (e.g., cardiometabolic, mechanical, psychosocial, medical/family history, weight loss history, etc.), traits (e.g., behavioral, lifestyle, personality, etc.), real life factors (e.g., lifestyle, barriers, challenges, responsibilities, stressors, values etc.), and other information including the user's microbiome, genetics, and metabolism. Tailoring of the plan may be based upon data/input from a proprietary questionnaire, user input (from various sources online, health-kits, devices etc.), and the user's health vision (which may, for example, be created using an integrative health methodology). In some embodiments, execution of these plans may then be followed in real-time, and be further personalized and modified, based on user input that includes physiological and psychosocial, data analytics, and human/virtual coaching feedback. For example, data collected in real time may relate to user compliance and adherence with aspects of his/her plan, biofeedback, and behavioral/lifestyle data. Personalization methods may include EMA (Ecological Momentary Assessment) to enable real-time personalization, applying various decision-making algorithms (e.g., using Artificial Intelligence, Machine Learning, and/or any other suitable techniques), analysis of user data generated during the program, and the user's health vision and other integrative health inputs.

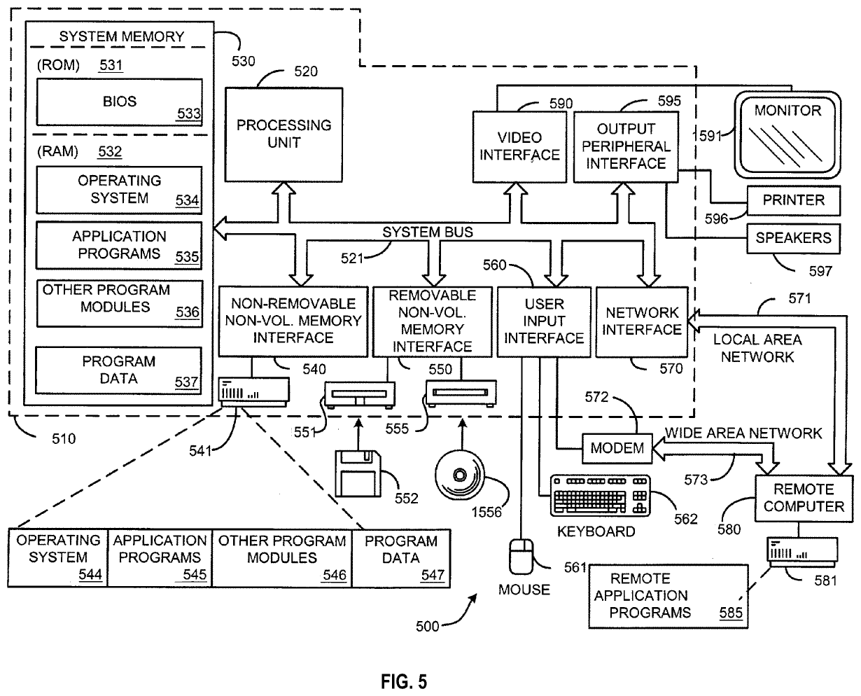

As noted above, some aspects of the invention may be implemented using a computing device. FIG. 5 depicts a general purpose computing device, in the form of a computer 410, which may be used to implement certain aspects of the invention. For example, computer 510 or components thereof may constitute any of the audio controllers, mobile devices, and/or networking components described above.

In computer 510, components include, but are not limited to, a processing unit 520, a system memory 530, and a system bus 521 that couples various system components including the system memory to the processing unit 520. The system bus 521 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 510 typically includes a variety of computer readable media. Computer readable media can be any available media that can be accessed by computer 510 and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other data. Computer storage media include, but are not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other one or more media which may be used to store the desired information and may be accessed by computer 510. Communication media typically embody computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared and other wireless media. Combinations of the any of the above should also be included within the scope of computer readable media.

The system memory 530 includes computer storage media in the form of volatile and/or nonvolatile memory such as read only memory (ROM) 531 and random access memory (RAM) 532. A basic input/output system 533 (BIOS), containing the basic routines that help to transfer information between elements within computer 510, such as during start-up, is typically stored in ROM 531. RAM 532 typically contains data and/or program modules that are immediately accessible to and/or presently being operated on by processing unit 520. By way of example, and not limitation, FIG. 5 illustrates operating system 534, application programs 535, other program modules 539, and program data 537.

The computer 510 may also include other removable/non-removable, volatile/nonvolatile computer storage media. By way of example only, FIG. 5 illustrates a hard disk drive 541 that reads from or writes to non-removable, nonvolatile magnetic media, a magnetic disk drive 551 that reads from or writes to a removable, nonvolatile magnetic disk 552, and an optical disk drive 555 that reads from or writes to a removable, nonvolatile optical disk 559 such as a CD ROM or other optical media. Other removable/non-removable, volatile/nonvolatile computer storage media that can be used in the exemplary computing system include, but are not limited to, magnetic tape cassettes, flash memory cards, digital versatile disks, digital video tape, solid state RAM, solid state ROM, and the like. The hard disk drive 541 is typically connected to the system bus 521 through an non-removable memory interface such as interface 540, and magnetic disk drive 551 and optical disk drive 555 are typically connected to the system bus 521 by a removable memory interface, such as interface 550.

The drives and their associated computer storage media discussed above and illustrated in FIG. 5, provide storage of computer readable instructions, data structures, program modules and other data for the computer 510. In FIG. 5, for example, hard disk drive 541 is illustrated as storing operating system 544, application programs 545, other program modules 549, and program data 547. Note that these components can either be the same as or different from operating system 534, application programs 535, other program modules 539, and program data 537. Operating system 544, application programs 545, other program modules 549, and program data 547 are given different numbers here to illustrate that, at a minimum, they are different copies. A user may enter commands and information into the computer 510 through input devices such as a keyboard 592 and pointing device 591, commonly referred to as a mouse, trackball or touch pad. Other input devices (not shown) may include a microphone, joystick, game pad, satellite dish, scanner, or the like. These and other input devices are often connected to the processing unit 520 through a user input interface 590 that is coupled to the system bus, but may be connected by other interface and bus structures, such as a parallel port, game port or a universal serial bus (USB). A monitor 591 or other type of display device is also connected to the system bus 521 via an interface, such as a video interface 590. In addition to the monitor, computers may also include other peripheral output devices such as speakers 597 and printer 599, which may be connected through a output peripheral interface 595.

The computer 510 may operate in a networked environment using logical connections to one or more remote computers, such as a remote computer 580. The remote computer 580 may be a personal computer, a server, a router, a network PC, a peer device or other common network node, and typically includes many or all of the elements described above relative to the computer 510, although only a memory storage device 581 has been illustrated in FIG. 5. The logical connections depicted in FIG. 5 include a local area network (LAN) 571 and a wide area network (WAN) 573, but may also include other networks. Such networking environments are commonplace in offices, enterprise-wide computer networks, intranets and the Internet.

When used in a LAN networking environment, the computer 510 is connected to the LAN 571 through a network interface or adapter 570. When used in a WAN networking environment, the computer 510 typically includes a modem 572 or other means for establishing communications over the WAN 573, such as the Internet. The modem 572, which may be internal or external, may be connected to the system bus 521 via the user input interface 590, or other appropriate mechanism. In a networked environment, program modules depicted relative to the computer 510, or portions thereof, may be stored in the remote memory storage device. By way of example, and not limitation, FIG. 5 illustrates remote application programs 585 as residing on memory device 581. It will be appreciated that the network connections shown are exemplary and other means of establishing a communications link between the computers may be used.

Embodiments of the invention may be embodied as a computer readable storage medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments of the invention discussed above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the present invention as discussed above. As used herein, the term “computer-readable storage medium” encompasses only a tangible machine, mechanism or device from which a computer may read information. Alternatively or additionally, the invention may be embodied as a computer readable medium other than a computer-readable storage medium. Examples of computer readable media which are not computer readable storage media include transitory media, like propagating signals.

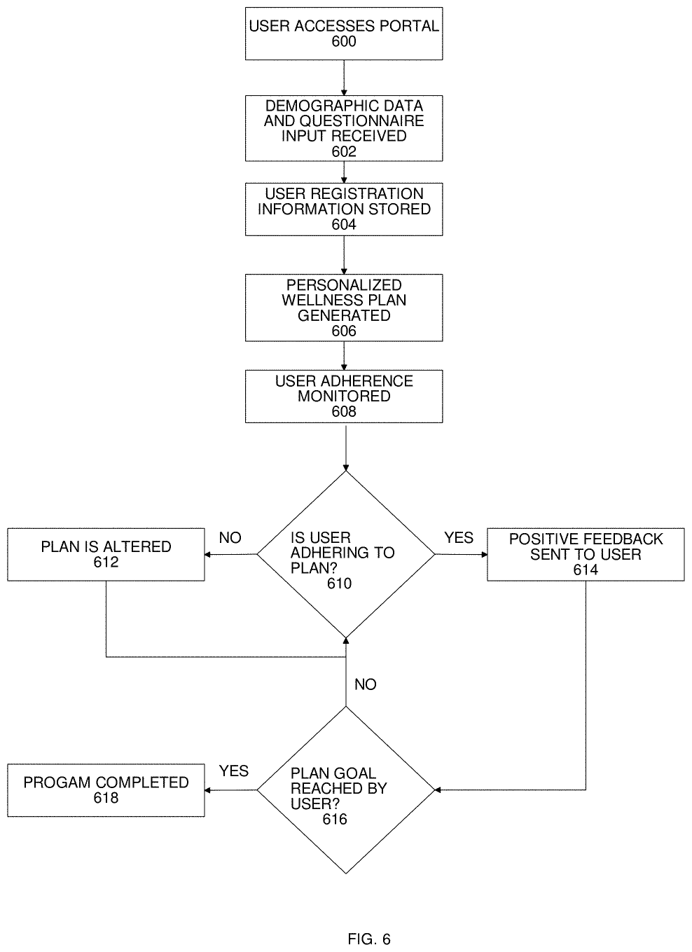

FIG. 6 is a flowchart for generated a personalized wellness plan and monitoring user adherence to the plan, in accordance with some embodiments of the invention. In step 600, the user may access the platform by supplying data like a user name, password, and basic demographic data. The portal can be located on a smartphone application downloaded onto the user's device, or alternatively, the portal can be access via a mobile-enabled or traditional Internet browser.

In step 602, the user can provide high-level demographic information, such as, for example, their gender, age, weight, height, BMI, and geographic location. In addition, the user can complete a risk assessment questionnaire and/or a proprietary questionnaire, as discussed in more detail above. In step 604, this information is then stored in a database, which may be of any suitable type. The information is processed by applying one or more decision-making algorithms, such as one or more rule-based and/or machine learning procedures. In step 606, the system generates and outputs a risk report that is personalized for the user, as well as a personalized wellness plan with respect to nutrition, behavior and activity. The personalized wellness plan can include, for example, meal and nutrition plans, exercise plans, sleep routine planning, lifestyle and behavior modification, etc.

In step 608, user adherence is continually monitored by the system. For example, the system can receive data from the user's wearable or mobile computing device, sleep tracking device, smart scale, etc., as well as data related to the user's laboratory and medical records. This information stored in the database is continually updated over time, and decision-making algorithms process the updated data to provide real-time, meaningful feedback with respect to nutrition, behavior and activity.

In step 610, the system determines if the user is adhering with the personalized wellness plan. With respect to nutrition, decision-making algorithms may define such aspects of a tailored nutrition plan as composition (e.g., food items composing the user's diet) and calories (e.g., for each food item). With respect to behavior, decision-making algorithms may define such aspects as the frequency and intensity of tailored behavior intervention. With respect to exercise, decision-making algorithms may define such aspects as exercise intensity and frequency (e.g., minutes per week). The continually updated information in the database is used to not only provide feedback to users, but to continually refine and train the decision-making algorithms, so that the algorithms may deliver more effective and personalized feedback over time.

Input supplied by the user (e.g., in signing up with the platform, and/or over time as the user engages with the platform to manage his/her weight) is stored in a database. Additionally, information regarding devices that the user customarily uses, including any wearables, is also stored in the database. Data in the database is used to generate reports as well as alerts, reminders and notifications. The reports, alerts, reminders and notifications may, for example, be sent both to the user and to the user's coach, so that the coach may provide observations, commentary and additional feedback to the user based on the information being sent to the user.

Decision-making algorithms (such as, for example, “deep learning/machine learning models”) are applied to data stored in the database, such as to discern a nutritional pattern by the user, the overall population, and/or by user-specific population cohorts. As a result of identifying these patterns, a personalized wellness plan may be initially developed for the user, and updated over time, such as to change the composition of the user's nutrition plan or the number of calories which the user is budgeted to consume in a particular time period.

The user supplies data such as mood, device usage, lab results, physiology information, compliance, geocode, behavioral, coaching and other information. This and/or other information is processed using one or more decision-making algorithms (e.g., a rule-based algorithm, as is known in the prior art to provide general, high-level feedback to the user, or a machine learning, deep learning, pattern recognition and/or artificial intelligence-based procedure to produce tailored feedback to the user). As an example, in response to the user reporting the composition of her lunch, real-time blood glucose data may be generated, and the amount of carbohydrates in the meal may be calculated.

If the system determines that the user is not adhering to the plan in step 610, the above information may be used alter the wellness plan for the rest of the day, and/or cause the remainder of the program plan for the user to be modified. Any modifications may, for example, be based on patterns observed in the user, in users determined to be similar in one or more respects, and/or in the overall population. Factors which may influence a decision to modify a specific user's nutrition plan may include the user's geocode, various characteristics of his/her environment, the user's physiology, physical activity, and/or other information. In addition, the system can display a visual graphic showing the user's progress towards their goal in order to provide motivation to adhere to the plan. In yet another embodiment, the user can be displayed an updated timeline for a new goal based on the modified wellness plan. The user can also receive feedback delivered to the user via their wearable device and/or mobile computing device indicating that they are not adhering to their plan.

If the system determines that the user is adhering to the plan in step 610, the user can receive positive feedback in step 614. The positive feedback can include an audio or video call from a virtual or live coach, or a text, email or MMS message. In addition, the positive feedback can include positive reinforcement, such as a visual graphic showing the user's progress and achievements towards their goal. The positive feedback can also include haptic feedback delivered to the user via their wearable device and/or mobile computing device.

In step 616, the system determines whether the user has reached their goal as per their personalized wellness plan. If the user has reached their goal, the program is deemed complete in step 618. In an embodiment, the system can provide guidance, instructions, motivations, tips, etc. to encourage the user to maintain their achieved goals (i.e., reduced weight, reduced blood pressure, etc.).

In an embodiment, the user can provide a listing of preferred foods or food types. The list can include a user's favorite foods, foods typically eaten by a user, favorite restaurants, etc. In another embodiment, the system utilizes machine learning to understand, over time, a dietary pattern of the user to determine certain foods or food types that the user prefers. For example, the system detects that the user eats cereal each morning at home, or stops at a bagel shop each morning on the way to work, such data can be used to determine a preferred food or food type for the user.

As part of the personalized wellness plan, the system generates a tailored list of food items identified for the user, based upon a target composition and calories, may include a list of food items to be consumed at breakfast, lunch, dinner and snacks. Each meal may include food items that are categorized as being prepared at home, at a cafeteria, at a restaurant, from a vending machine, packaged, or backup.

In some embodiments, a user's personalized wellness plan may be accessible via a portal that is made available to not only the user, but also the user's coach. A coach may implement protocols in interacting with a user, such as via on-line and/or automated means, in person, or via video or audio. The user and/or coach select up to three choices for each eating episode, and the user may choose his/her food from these choices. In addition, the choices can be based on, for example, patterns observed during previous eating sessions, such as to modify glucose levels, carbohydrate intake, protein intake, etc. If one of the three choices is not ultimately selected by the user for an eating episode, he/she may enter text and/or upload a picture of the food item that is actually consumed, and image recognition technology may be used to identify the food item, its composition and nutrients, and/or other information so that immediate feedback may be provided on whether the item complies with the user's nutrition plan.

The system can further determine, as part of the personalized wellness planning process, if any of a user's preferred foods or food types is allowed, or if they should be restricted, based on machine learning that utilizes the user's physiological data. For example, physiological data, such as the user's weight, body mass index, metabolism, gut microbiome, stress level, or epigenetics, or a health condition selected from a group consisting of obesity, diabetes, chronic disease, cardiovascular disease, and hypertension, can be used to determine if a preferred food or food type would worsen or improve the physiological data.

In another embodiment, the system can determine, as part of the personalized wellness planning process, if any of a user's preferred foods or food types is allowed, based on an analysis of a population cohort having similar physiological data as the user.

A coach may also work with a user to establish timing for each eating episode, and in establishing notifications in support of this timing. The notifications may be default, or personalized to the user. The coach may also work with the user to establish ways to gauge their hunger level, and determine eating episode timing based at least in part on this information.

In some embodiments, the platform may employ feedback rules that govern how data supplied by the user and/or coach is used at designated time intervals to generate user notifications. For example, a rules engine may process data provided by the user and/or coach to generate messages which are based on positive and negative outcomes, flags, and coach notifications, and progress tracking (e.g., of goals, certain parameters, compliance and adherence, etc.).

In some embodiments, a personalized wellness plan may account for not only food items, but also beverages in the user's diet. For example, the platform may monitor and provide feedback on non-caloric fluid intake, sugar sweetened beverage intake (when applicable), and alcoholic beverage intake.

Input supplied to a device, such as a wearable device, may comprise affirmative input provided by the user and/or information captured without the user having to affirmatively supply it. The input may be stored in a database, and used to generate updated information for his/her coach, progress notes, protocols and data. One or more decision-making algorithms may process the input to generate notifications, feedback, alerts and reminders, and reports for the user. This information may also be sent to the user's coach, along with flags that may be useful in providing feedback to the user.

Information supplied by the user may also be used to generate reports relating to nutrition, activity, goals, etc. For example, the information may be processed to generate analytics, determine patterns, and/or derive actionable insights. A rules engine may send such feedback to the user as an action item, a modified food list for the user, and/or any other suitable feedback.

As noted above, in some embodiments, the platform may enable the user to get instant feedback on food items encountered by the user, such as to determine whether the food items comply with the user's nutrition plan. For example, if the user is at a restaurant (e.g., as determined using a GPS component of a device worn or transported by the user), the user may take and upload a picture of a food item. Image recognition technology may be used to identify the item, apply one or more rules specified by a food database, and generate feedback that may be provided to the user in real time regarding whether the food item complies with the user's nutrition plan. The user can receive feedback via the software application, or via human contact by their coach after submitting the food item information. For example, the coach may call, text, message, and/or initiate a video conference with the user in real-time to reinforce that the proposed food item is not an optimal or ideal choice to reach the user's weight loss and wellness goals, and the coach can assist the user in real-time in selecting another option.

Information supplied by the user may be processed to determine a behavioral plan defining, for example, the frequency and intensity of behavioral intervention. For example, a personalized behavior plan may define the frequency of one-on-one coaching sessions, which may relate to the user's lifestyle, integrative health needs, and/or other aspects of the user's behavior. Automated procedures may be executed to identify behavioral traits, which may drive the intensity with which behavioral intervention is performed. For example, a learning management system (LMS) may assign evidence-based modules to each user that may be modeled after “one size fits all” programs, but be tailored to the user's specific situation. One or more decision-making algorithms may, for example, be applied to data supplied by the user, his/her coach, his/her healthcare provider, and/or other data sources to define a personalized behavior plan.

The information on which such feedback is based may include data supplied by the user (e.g., the indication of the user's vision and values supplied during initial platform access), and data recorded by the user's coach in progress notes during consultations. This information may be stored in the database, and processed to produce reports and analytics, as well as flags for a rules engine that may produce virtual feedback and updated progress reports. Data stored in the database may also be processed to produce flags for the user's coach which may be employed to produced personalized notifications to the user, and possible program modifications if the user approves. In some embodiments, the behavioral principles employed in providing feedback to the user may include self-monitoring, self-efficacy, stimuli narrowing, cognitive restructuring and stimuli control principles.

In the example shown, one or more decision-making algorithms (e.g., artificial intelligence, machine learning or deep learning procedures) may process biofeedback from a device worn or used by the user, such as a wearable device or a wireless scale. Data collected from one or more devices may be used to generate notifications, alerts and reminders to the user, and any actions taken by the user as a result of the feedback may be processed by a rules engine and supplied to the user's coach for use in modifying the user's tailored behavioral program, if the user approves. For example, an increase in the user's heart rate (e.g., caused by stress, exercise, anxiety or some combination thereof) may be correlated with the user's GPS coordinates (e.g., indicating that the user is then at work) to generate personalized feedback to the user. For example, integrative health and mindfulness principles may be utilized to reassess the user's health vision and values, and to apply mindfulness skills in producing feedback. In this respect, integrative health principles may be incorporated into an overall curriculum for the user, such as by strategically placing integrative health sessions amongst regular lifestyle coaching sessions. For example, mindfulness based self-awareness may be used to ground the user in the moment's reality and problem solve using data (e.g., visual data) and report-based from platform, coaching and other materials (e.g., curriculum, meditation audio, videos, etc.).

Information supplied by the user via the website is processed using one or more decision-making algorithms to generate a personalized activity plan which specifies the intensity and frequency of activity for the user. In some embodiments, a personalized activity plan may include a tailored exercise regimen, videos, locations for training, and other plan specifics. Further, some embodiments of the invention may provide for generating and sending notifications to the user relating to the exercise plan. For example, the user's actual activity may be tracked using data received from devices transported and/or worn by the user. In some embodiments, information produced by a device in tracking whether the user conforms to his/her personalized exercise plan may be processed to produce feedback to the user, and to implement modifications to the user's exercise plan going forward.

Information from devices which measure metabolism (which changes as an individual loses weight) may be stored to a database and processed to produce feedback indicating, for example, that the user should increase the number of minutes per week that he/she exercises, and/or increase the intensity of such exercise to stay on track to achieve his/her goals. FIG. 10 depicts another example in which a wireless scale provides information on a user's weight and/or body mass index (BMI), so that the user's adherence to his/her personalized activity plan may be assessed, corresponding feedback to the user may be generated, and coaching may be administered as appropriate.

One or more decision-making algorithms may be used to process data relating to a user to perform initial tailoring, define personalized nutrition, behavior and activity plans, and define rules for a user-specific curriculum. The data to which the decision-making algorithms are applied may be collected using any suitable collection of components, in any suitable way. Further, the computing system(s) on which the decision-making algorithms execute may comprise any suitable collection of hardware and software components.

In some embodiments, a platform may comprise a website or app through which a user accesses and/or interact with the platform, one or more food databases, a learning management system, and a food recognition API. Of course, a platform implemented in accordance with some embodiments of the invention may comprise other software components, as the invention is not limited in this respect.

Any of numerous hardware devices may be used to collect data on user nutrition, behavior and activity. Representative hardware devices may include a wireless scale, one or more wearable devices adapted to be worn by the user, a hand-held RMR, and one or more genetics/microbiome kits. Of course, any suitable hardware devices may be used to collect data on users, as the invention is not limited in this respect.

In some embodiments of the invention, platform components may facilitate interaction with the user during an initial (e.g., six month) period during which he/she receives substantially continuous feedback relating to nutrition, behavior and exercise. At the end of this initial period, an assessment may be performed to determine whether the user's goals (e.g., defined at the outset of the initial period) have been met. If so, the user may be transitioned to a weight maintenance plan, which may proceed in periodic (e.g., six month) cycles. If the user has not met his/her goals, then the plan initially defined may be modified as needed, based on various inputs, including user, hormonal, physiological (RMR), IHC, and other inputs. These inputs may be processed by applying one or more decision-making algorithms, such as artificial intelligence, machine learning and/or pattern recognition procedures, to develop a modified plan for a follow-on period.

As noted above, some embodiments of the invention may apply decision-making algorithms in processing data relating to a user. For example, some embodiments may apply decision-making algorithms in processing data indicating internal and external factors affecting the user. The algorithms may be used to track the user's progress, provide personalized real-time feedback, and modify or refine the user's therapeutic plan, to enable a tailored, personalized, and precision based high-intensity comprehensive lifestyle intervention for medical management of weight and obesity.

In some embodiments, decision-making algorithms may be used to track a user's nutritional intake, generate personalized real-time nutrition feedback, and cause changes to the user's nutrition plan based on information such as data on special circumstances (e.g., travel, weight regain, injury preventing exercise, life stressors), physiology (e.g., hunger/satiety hormone levels, metabolic rate (which may change as the user loses weight), behavioral health (e.g., assessed using psychometric scales and/or other techniques), gut microbiome (which may also change as a user loses weight), and/or other information.

In some embodiments, decision-making algorithms may be used to track a user's ongoing exercise and activity, generate personalized real-time activity feedback, and cause changes to the user's activity plan based on information including data gathered from a device worn or transported by the user, the user's metabolic rate, external factors (e.g., life stressors, weather, time management, health, etc.) and/or other information.

In some embodiments, decision-making algorithms may be used to track a user's behavior, generate personalized real-time behavioral feedback, and cause changes to the user's behavioral intervention plan based on information including data on the user's eating, activity, and lifestyle related behaviors, external and internal factors (e.g., hormones, fatigue, low metabolism, etc.), and/or other information.

Feedback which is generated through application of decision-making algorithms may be provided to not only the user, but also the user's coach, to optimize program results, compliance, and adherence. Feedback may be personalized, delivered in real time, and designed to encourage the user and/or coach to make appropriate nutritional, activity and/or behavioral changes as needed.

In some embodiments, decision-making algorithms may be used to detect noise in data relating to a user or population of users. Further, decision-making algorithms may be used to detect and address individual variability in response to treatment. In this respect, some factors which may affect treatment response may be known at any given time to a healthcare provider or a coach, but many may be unknown, or it may be too later after a treatment period has finished to address issues retrospectively. As one example, insulin response to carbohydrate load (high amount of sugar/starch) in a meal is diminished in post-menopausal women, but there may be individual variability. A clinician may not know the carb threshold for a given user, but through the application of decision-making algorithms, it may be possible to determine the amount of carbohydrate needed to lose or maintain current weight. As another example, individuals may vary with respect to exercise tolerance, and some users may develop an inflammatory response to exercise and need to reduce activity, while others may not be exercising efficiently despite spending a recommended amount of time in the gym. Through the application of decision-making algorithms, it may be possible to detect reduced user tolerance to exercise and modify plans accordingly. As yet another example, decision-making algorithms may enable the prediction of behavioral episodes (e.g., binge eating episodes, and other eating disorder-related acute episodes), and allow behavioral therapy to be modified accordingly, and a user's coach and/or healthcare or mental health provider to be alerted, if appropriate.

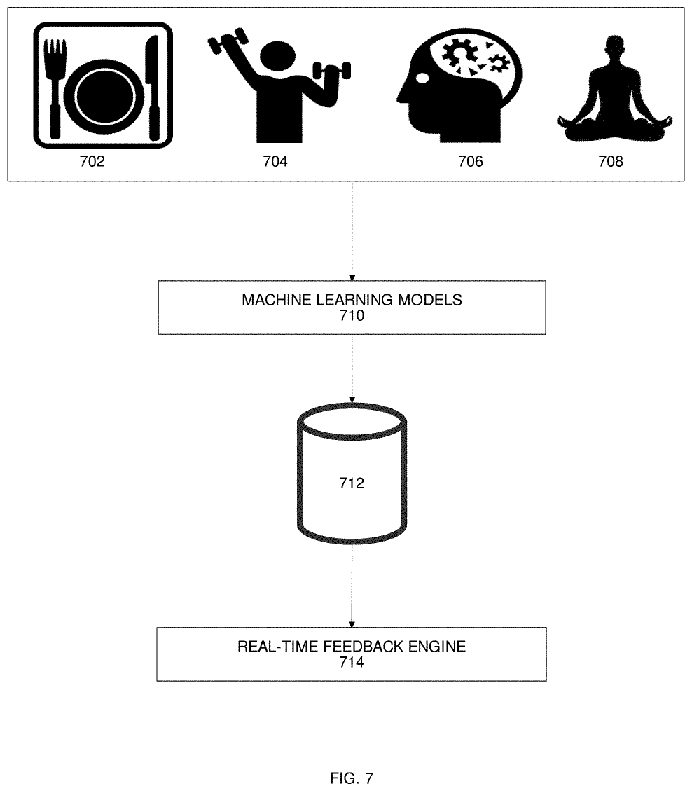

FIG. 7 is a diagram depicting the various aspects of personalized wellness plan. The personalized wellness plan can include various programs, such as a tailored nutrition and meal planning program 702, a tailored exercise and workout program 704, a tailored behavior and lifestyle modification program 706, and/or a tailored integrative health and mindfulness program 708. Each of the specific programs can be generated to be complimentary with one another. In an embodiment, the user can choose which specific program(s) they would like generated for their personalized wellness plan.

The user's frequency and intensity are monitored by the system to understand the user's adherence to each program, as well as the user's ability to complete the plan or specific tasks in each program. As discussed in greater detail above, machine learning models specific to each type of program can be employed to further fine-tune the user's personalized wellness plan, and to feed a real-time feedback engine 714 that provides feedback to the user regarding adherence, progress towards a goal, encouragement, and support. As discussed above, the feedback can be virtual, message-based, haptic, and/or provided by a human coach. In addition, the feedback can be in the form of notifications, alerts, reminders, and progress reports. In addition, reports such as caloric intake, meal summaries (i.e., weekly or daily summaries), activity reports, etc. can be generated by the real-time feedback engine 714.

It should be appreciated that a platform implemented in accordance with some embodiments of the invention does not merely automate previously employed approaches to obesity and weight management, and that the platform enables insights which would not have been possible using prior approaches, even given infinite computational resources and time. In this respect, a traditional approach to obesity and weight management, whether administered online or offline, is exclusively rule-based, and entirely dependent on user input. As a result, individualized, real-time feedback and program modification based on external and internal factors experienced by a user is not possible in a system using the traditional approach. (In this respect, factors which are internal (i.e., inside the user) include physiology, phenotype, genotype, epigenetics, behavior, user compliance, weight change, metabolism, gut microbiome, and internal stressors, and factors which are external (i.e., outside the person) include the user's environment, geocodes, food availability, and external stressors, including work, family, temperature, etc.). A traditional rule-based system cannot adapt to small or large changes in a user's internal and external environment, and/or give appropriate feedback to the user and a coach, or modify the user's program in real time. In a traditional approach, a single user's pattern cannot be compared with specific population cohorts (e.g., other users with diabetes, others with jobs requiring travel, users experiencing similar stresses, etc.) in real time. Additionally, signal to noise detection and feedback based on minor deviations is not possible using a traditional approach.

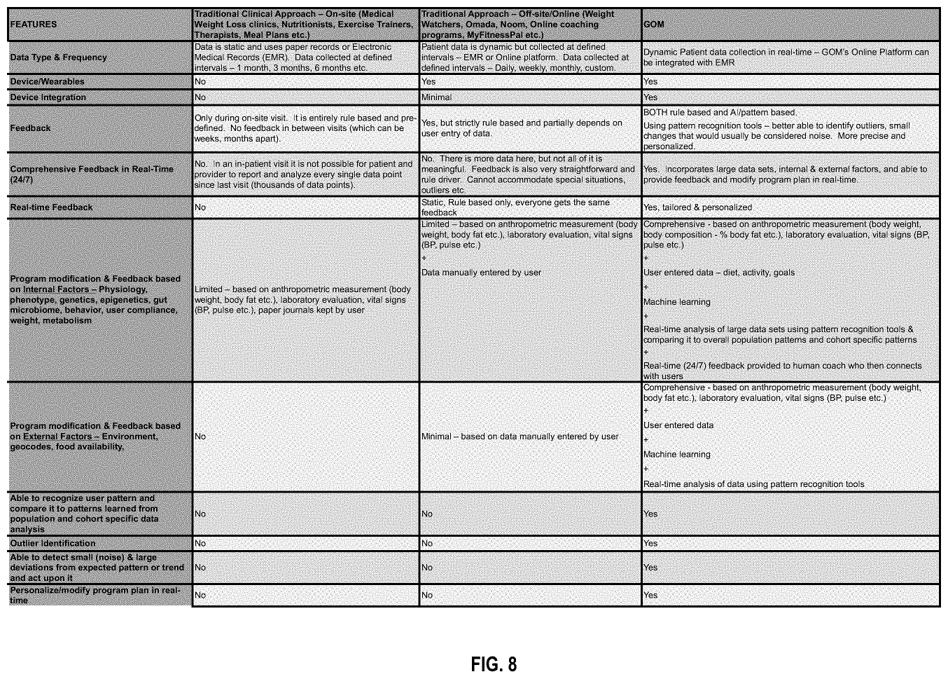

A summary of the differences between a traditional clinical approach, a traditional online approach, and an approach in accordance with embodiments of the invention (labeled “GOM”) is shown in FIG. 8.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Further, though advantages of the present invention are indicated, it should be appreciated that not every embodiment of the invention will include every described advantage. Some embodiments may not implement any features described as advantageous herein and in some instances. Accordingly, the foregoing description and drawings are by way of example only.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

The invention may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those which are described, and/or which may involve performing some acts simultaneously, even though the acts are shown as being performed sequentially in the embodiments specifically described above.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

While the principles of the disclosure have been illustrated in relation to the exemplary embodiments shown herein, the principles of the disclosure are not limited thereto and include any modification, variation or permutation thereof.

What is claimed is:

1. A method for monitoring adherence to a behavior modification program, comprising:
receiving, at a server, a preferred food type of a user and a physiological data related to the user;
determining, by the server, if the preferred food type is a restricted food or an allowed food, based on the physiological data;
receiving, at the server, location data collected over a period of time from at least one of a wearable device or a mobile computing device;
determining, by the server, a user's commute based on the location data;
identifying, by the server, a dining option along the commute that offers the preferred food type;
transmitting, by the server, a warning message to the user to avoid dining at the dining option if the preferred food type is a restricted food; and
transmitting, by the server, an encouragement message to the user to dine at the dining option if the preferred food type is an allowed food.

2. The method of claim 1, wherein the physiological data is related to at least one of the user's weight, body mass index, metabolism, gut microbiome, or epigenetics.

3. The method of claim 1, wherein the physiological data indicates at least one health condition selected from a group consisting of obesity, diabetes, chronic disease, and cardiovascular disease.

4. The method of claim 1, wherein the server determines if the preferred food type is a restricted food or an allowed food using a machine learning technique.

5. The method of claim 1, wherein the warning message is displayed on a display of the wearable device or the mobile computing device.

6. The method of claim 1, wherein the warning message is a live audio or video call from a human coach or a virtual coach.

7. The method of claim 1, further comprising, transmitting, by the server, a list of alternative dining options if the preferred food type is a restricted food.

8. A method for monitoring adherence to a behavior modification program, comprising:
receiving, at a server, a preferred food type of a user and a physiological data related to the user;
determining, by the server, if the preferred food type is a restricted food or an allowed food, based on the physiological data;
receiving, at the server, location data collected over a period of time from at least one of a wearable device or a mobile computing device;
determining, by the server, a user's commute based on the location data;
identifying, by the server, a dining option along the commute that offers the preferred food type;
analyzing, by the server, a hunger hormone level of the user;
transmitting, by the server, a warning message to the user to avoid dining at the dining option if the preferred food type is a restricted food and the hunger hormone level of the user is above a threshold value; and
transmitting, by the server, an encouragement message to the user to dine at the dining option if the preferred food type is an allowed food.

9. The method of claim 8, wherein the hunger hormone level is based on analysis of a hormone selected from a group consisting of ghrelin, leptin, cortisol, glucose, insulin, neuropeptide Y (NPY), agouti-related protein (AgRP), proopiomelanocortin, alpha-melanocyte stimulating hormone (α-MSH), cocaine- and amphetamine-regulated transcript (CART), cholecystokinin, peptide tyrosine tyrosine (PYY), pancreatic polypeptide (PP), oxyntomodulin, glucagon-like peptide 1 (GLP-1), gastric inhibitory polypeptide (GIP), and adiponectin.

10. The method of claim 8, wherein the server determines if the preferred food type is a restricted food or an allowed food based on an analysis of a population cohort having a similar physiological data as the user.

11. The method of claim 8, wherein the physiological data indicates a stress level of the user.

12. The method of claim 8, wherein the warning message includes a weight loss goal.

13. The method of claim 8, wherein the encouragement message includes a visual indication of progress towards a weight loss goal.

14. The method of claim 8, where the warning message includes a haptic feedback delivered via the wearable device or the mobile computing device.

15. A method for monitoring adherence to a behavior modification program, comprising:
receiving, at a server, a preferred food type of a user and a physiological data related to the user;
determining, by the server, if the preferred food type is a restricted food or an allowed food, based on the physiological data;
receiving, at the server, location data collected over a period of time from at least one of a wearable device or a mobile computing device;
determining, by the server, a user's commute based on the location data;
identifying, by the server, a dining option along the commute that offers the preferred food type;
analyzing, by the server, a hunger hormone level of the user;
transmitting, by the server, an alternative commute for the user in order to avoid being in proximity to the dining option if the preferred food type is a restricted food and the hunger hormone level of the user is above a threshold value.

16. The method of claim 15, wherein the server determines the alternative commute based using a machine learning technique.

17. The method of claim 15, wherein the server determines if the preferred food type is a restricted food or an allowed food using a machine learning technique.

18. The method of claim 15, wherein the hunger hormone level is based on analysis of a hormone selected from a group consisting of ghrelin, leptin, cortisol, glucose, insulin, neuropeptide Y (NPY), agouti-related protein (AgRP), proopiomelanocortin, alpha-melanocyte stimulating hormone (α-MSH), cocaine- and amphetamine-regulated transcript (CART), cholecystokinin, peptide tyrosine tyrosine (PYY), pancreatic polypeptide (PP), oxyntomodulin, glucagon-like peptide 1 (GLP-1), gastric inhibitory polypeptide (GIP), and adiponectin.

19. The method of claim 15, wherein the server determines if the preferred food type is a restricted food or an allowed food based on an analysis of a population cohort having a similar physiological data as the user.

20. The method of claim 15, wherein the physiological data indicates at least one medical condition selected from a group consisting of obesity, diabetes, chronic disease, and cardiovascular disease.
